(19) United States
(12) Patent Application Publication
(10) Pub. No.: US 2023/0417674 A1
(43) Pub. Date: Dec. 28, 2023

(54) IN-VITRO MEDICAL DIAGNOSTIC DEVICE AND IN-VITRO MEDICAL DIAGNOSTIC SYSTEM

(71) Applicant: EDAN INSTRUMENTS, INC., Shenzhen (CN)

(72) Inventors: Gaoxiang HUANG, Shenzhen (CN); Zhixiang ZHAO, Shenzhen (CN)

(21) Appl. No.: 18/343,719

(22) Filed: Jun. 28, 2023

Related U.S. Application Data
(63) Continuation of application No. PCT/CN2021/143216, filed on Dec. 30, 2021.

(30) Foreign Application Priority Data
Dec. 31, 2020 (CN) 202011629633.0

(51) Int. Cl. G01N 21/64; G01N 33/72; G01N 35/10

(52) U.S. Cl. CPC G01N 21/6428; G01N 33/726; G01N 35/1016; G01N 2201/08

(57) ABSTRACT

An in-vitro medical diagnostic system includes a host (1), a removable test card (2) and a removable reagent pack (3). The reagent pack (3) includes a calibrating liquid needed in a process for calibrating the test card (2); the reagent pack (3) is clamped to the host (1), with only first power drive and valve control transmission being provided between the host (1) and the reagent pack (3); and the test card (2) is mounted on the host (1) during the entire test process, with only second power drive and an optical conduction path being provided between the host (1) and the test card (2). No liquid path is present in the host (1) of the in-vitro diagnostic system, thus eliminating hidden medical dangers. Tests for blood gas, hemoglobin and derivatives thereof can be achieved in a single test card.

### CROSS-REFERENCE TO RELATED APPLICATIONS

The present application is a continuation of International (PCT) Patent Application No. PCT/CN2021/143216 filed on Dec. 30, 2021, which claims priority to Chinese patent application No. 202011629633.0, filed on Dec. 31, 2020, the contents of all of which are hereby incorporated by reference.

### TECHNICAL FIELD

The present disclosure relates to the field of in-vitro technologies, and more particularly, to an in-vitro medical diagnosis device and an in-vitro medical diagnosis system.

### BACKGROUND

The determination of gas component in blood is very important in various scientific researches and practical applications. In the rescue of critical clinical medicine patients, rapid and continuous determination of the partial pressure of blood carbon dioxide is critical. Especially for mechanically ventilated patients, blood carbon dioxide partial pressure is a key index for judging respiratory state of patient, and various parameters of the breathing machine are mainly set according to the partial pressure of carbon dioxide in the blood of the patient. At present, the most widely used blood gas component detector in medicine is a blood gas analyzer, but the conventional blood gas analyzer has the defects that a large number of blood samples need to be collected, the detection is discontinuous, the detection result is lagged, and the like.

The traditional in-vitro blood gas detection is performed in a large test center with good equipment. Although these conventional test centers can provide efficient and accurate testing of large volume fluid samples, it is not possible to provide a direct result. The practitioner must collect the fluid sample, send it to the laboratory, then the fluid sample is processed by the laboratory, and finally, the result is conveyed the patient. This traditional detection method causes a long and complex cycle of blood gas testing, it is difficult for patients to obtain timely diagnostic results, which is not conducive to timely diagnosis by medical staff and cannot provide patients with a good medical experience.

In addition, conventional in-vitro diagnostic testing requires training laboratory technicians to perform the test, thereby ensuring the accuracy and reliability of the test. However, usage errors caused by personnel handling samples may lead to surface contamination, sample spillage, or damage to diagnostic devices, resulting in increased repair and maintenance costs.

At the same time, in the related art, in-vitro medical diagnostic systems are generally complex in construction, particularly, a liquid path is inside of the host of the in-vitro medical diagnosis system. If the in-vitro medical diagnosis system is not maintained in time after long-term use, the leakage of the liquid path in the host is easily present, the accuracy of in-vitro diagnosis is affected, and serious potential safety and health hazards are caused.

At the same time, in the in-vitro diagnostic apparatus of the related art, if only one test card is used to detect the blood of the patient, only blood gas detection can be completed, or only hemoglobin detection can be completed. When the blood of the patient needs to detect the indexes of blood gas and hemoglobin at the same time, it is often necessary to take two copies of the patient's blood. This is not a common problem for healthy adults. However, for newborns or patients with less blood volume (e.g., anemia), various inconveniences may be brought to the patient, and even for healthy adults, if indexes of blood gas and hemoglobin need to be detected at the same time, two teat cards need to be used, so that the diagnosis cost of the patient is greatly increased, and resource waste may be caused.

At the same time, in the related art, it is often necessary to calibrate the sensors in the test card before the test card is used, therefore, in order to save cost, the calibrating liquid needed by calibration is usually stored in the test card. However, the consequent problem is that if the calibration liquid is stored in the detection card, in order to ensure that the calibration liquid cannot be affected by severe environmental factors such as air temperature and humidity, the test card has higher requirements on the storage condition and the environment in the transportation and storage processes, so that the calibration liquid in the test card is prevented from being affected, and further the calibration of the test card sensor is prevented from being affected.

### SUMMARY OF THE DISCLOSURE

An in-vitro medical diagnostic device includes a housing, a first mounting area configured to mount a removable reagent pack, a second mounting area configured to mount a removable test card, an optical system configured for in-vitro diagnosis and being inside of the device, and a power drive system configured to control at least on-off of a pipeline in the test card and/or to control a flow of a liquid in the reagent pack. The power drive system is inside of the device, and the optical system is at least configured to detect a blood gas, a hemoglobin and/or an electrolyte in the test card.

In some embodiment, the in-vitro medical diagnostic device further includes a positioning attachment area configured to connect an interface of the reagent pack and an interface of the test card.

In some embodiment, the optical system has one or more light source systems.

In some embodiment, at least one first light source system configured to activate a photochemical sensor in the test card is disposed below the second mounting area.

In some embodiment, an excitation light emitted by the first light source system passes through a first optical path and enters one end of an optical fiber bundle, and the other end of the optical fiber bundle is provided with an optical element configured to focus and illuminate the photochemical sensor in the test card.

In some embodiment, the first optical path includes at least a first lens, a first filter, and a first beam splitter.

In some embodiment, wherein the fiber optic bundle receives a fluorescence and/or reflected light generated in the test card, and transmits the fluorescence and/or reflected light to the optical detection element through a second optical path, and the second optical path is at least partially different from the first optical path.

In some embodiment, at least one second light source system is disposed above the second mounting region and configured to detect the hemoglobin and derivatives thereof and/or detect a flow position of an internal liquid of the test card.

In some embodiment, an excitation light emitted from the first light source has a central wavelength of 450 nm to 470 nm.

In some embodiment, a central wavelength of the fluorescent and/or reflected light is 520 nm to 650 nm.

In some embodiment, the optical system includes at least two light sources distributed on different sides of the second mounting area.

In some embodiment, the power drive system further includes a first power part configured to control the on/off of the pipeline in the test card.

In some embodiment, the power drive system further includes a second power part configured to control switching of the pipeline and liquid flow in the reagent pack.

In some embodiment, the second power part includes a first motor configured to control switching of a three-way valve in the reagent pack and a second motor configured to control rotation of a peristaltic pump in the reagent pack.

In some embodiment, the power drive system further includes a third power part configured to lock and unlock the test card.

In some embodiment, the power drive system outputs power in one or more mode of mechanical transmission, electricity, magnetism, and light.

In some embodiment, the in-vitro medical diagnostic device further includes one or more of a display element configured to display a diagnostic result, a printing element configured to output the diagnostic result, a bar code/two-dimensional code reading element, a battery, a UPS power supply.

An in-vitro medical diagnosis system includes a blood gas analyzer host, a removable test card, and a removable reagent pack. The reagent pack and the test card are connected through respective interfaces, and the device completes calibration, sample detection and emptying by providing power for the reagent pack and controlling on-off of a pipeline of the test card.

In some embodiment, the test card includes at least a blood gas detection area and a hemoglobin and derivatives detection area.

In some embodiment, a detection in the hemoglobin and derivatives detection area is implemented by colorimetric, electrochemical, or hemolytic method.

In some embodiment, the reagent pack includes at least one liquid storage device including an output pipeline, at least one transport control device configured to control at least a flow direction in a pipeline inside of the reagent pack, and at least one positioning mechanism configured to enable the transport control device to be driven by the system.

In some embodiment, a pipeline between the blood gas detection region and the hemoglobin and derivatives detection area is on-off connectable.

In some embodiment, the device controls on-off of the pipeline of the test card through a power system such that at least blood gas detection and hemoglobin detection is performed on the same blood sample or on different blood samples.

In some embodiment, the reagent pack at least includes calibration liquid required for one or more calibrations of the test card.

In some embodiment, an elastomeric material and/or valve is inside or on a surface of the test card, and the elastomeric material and/or the valve is configured to implement an on-off connection of the pipeline at a corresponding position of the test card.

In some embodiment, the test card includes at least three external interfaces; and the at least three external interfaces include a first interface configured to inject a calibration liquid, a second interface configured to inject a sample, and a third interface connected to a waste liquid area of the test card.

In some embodiment, the test card includes at least three pipeline control parts.

In some embodiment, the at least three pipeline control parts are respectively configured to control an on-off connection of the pipeline between the blood gas detection area and the hemoglobin and derivatives detection area, the on-off connection between a sample inlet of the test card and a liquid path inside of the test card, and the on-off connection between the liquid path inside of the test card and the waste liquid area of the test card.

In some embodiment, an electrical coupling connection is between the device and the test card.

In some embodiment, the electrical coupling connection is configured to supply power to an electrochemical sensor in the test card by the device.

### BRIEF DESCRIPTION OF THE DRAWINGS

FIG. 1 is a schematic view of an external structure of an in-vitro medical diagnostic system.

FIG. 2 is a schematic side view of the in-vitro medical diagnostic system.

FIG. 3 is a schematic view of a combination of components of the in-vitro medical diagnostic system.

FIG. 4 is a schematic semi-sectional view of a removable test card.

FIG. 5 is another schematic semi-sectional view of the removable test card.

FIG. 6 is yet another schematic semi-sectional view of the removable test card.

FIG. 7 is a schematic view of the connection of the removable test card and a valve control device.

FIG. 8A is a schematic control view of the valve control device.

FIG. 8B is another schematic control view of the valve control device.

FIG. 8C is yet another schematic control view of the valve control device.

FIG. 8D is yet another schematic control view of the valve control device.

FIG. 9 is a schematic cross-sectional view of a removable reagent pack.

FIG. 10 is a schematic view of an external structure of the removable reagent pack.

FIG. 11 is a schematic view of a body and a decoration cover of the removable reagent pack.

FIG. 12A is a schematic view of an excitation light source of the in-vitro medical diagnostic system.

FIG. 12B is another schematic view of an excitation light source of the in-vitro medical diagnostic system.

### DETAILED DESCRIPTION

It is necessary to design an in-vitro medical diagnosis system which is simple and compact in structure and safe and reliable, ensure that no hidden danger of liquid path leakage exists in the host in the system. At the same time, it can ensure that the calibration during detection is accurate and reliable, ensure that the detection of blood gas and hemoglobin index are completed in one test card. The more ideal condition is that the same blood sample is utilized to complete the detection of blood gas and hemoglobin indexes in one test card, and the transportation and storage links of the test card have low requirements on the storage condition and environment, and can ensure the accurate and reliable detection precision.

In view of the foregoing, a purpose of the present disclosure is providing a removable reagent pack for use in an in-vitro diagnostic device, and the removable reagent pack is capable of at least partially mitigating or eliminating at least one defect in the related art.

The present disclosure will now be further described in conjunction with the accompanying drawings and specific embodiments.

Before turning to the drawings illustrating exemplary embodiments in detail, it is to be understood that the present disclosure is not limited to the details or methods shown in the specification or illustrated in the drawings. The purpose of professional terminology is only for illustration and should not limit the understanding of present product and corresponding methods.

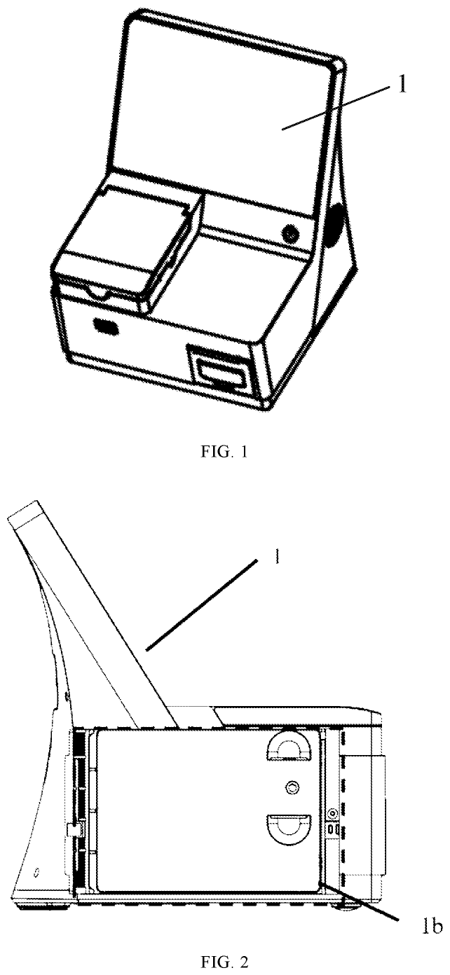

Exemplary embodiments of the present disclosure provide an in-vitro medical diagnostic system. As shown in FIGS. 1-3, the in-vitro medical diagnostic system includes a host 1, removable test card 2, and a removable reagent pack 3. The host 1 includes a housing, and processing circuitry, a power supply circuitry and optical elements located in the housing. The housing further includes a first area 1a configured to at least partially accommodate the removable test card 2, and a second area 1b configured to at least partially accommodate the removable reagent pack 3. The removable test card 2 is jointed with the host 1 and the removable reagent pack 3 through the first area 1a and the second area 1b, respectively, such that there is no fluid communication between the removable test card 2 and the host 1, and there is no fluid communication between the removable reagent pack 3 and the host 1.

Removable Test Card

The removable test card 2 includes an external interface, a detection area, a waste liquid area, a liquid path control area and an internal liquid path.

The external interface refers to a first interface for receiving fluid samples, a second interface for air pump inlet/outlet, and a third interface for injecting calibration liquid located on the sides, top, or bottom of the test card 2. Specifically, a fluid sample inlet is on the first side of the test card 2. The fluid sample inlet is configured to receive fluid samples, such as whole blood samples that do not require hemolysis. A calibration liquid inlet and an exhaust port are on the second side of the test card 2. The calibration liquid inlet is configured to receive calibration liquid from the outside (such as the removable reagent pack 3) for calibration of various photochemical sensors inside the test card 2. The exhaust port is configured to be connected to the external atmosphere to maintain pressure balance in the test card 2, in order to ensure that the calibration liquid or fluid sample flows into the test card 2.

The detection area refers to the area where electrical, optical, chemical sensors and/or cavities without sensors are placed for detecting various blood gas parameters. The detection of blood gas, hemoglobin, electrolytes, and other biochemical parameters is completed in this area.

The waste liquid area refers to the area configured to store the detected fluid samples.

The internal liquid path refers to the pipeline path configured to allow the fluid sample or calibration liquid to flow inside of the test card 2. The pipeline path has multiple interconnected liquid paths, and the detection area, the waste liquid area, and the liquid path control area are located on different liquid paths.

The liquid path control area refers to the area where several on-off control devices are located. The on-off control devices are configured to control the on-off of the internal liquid path, thereby achieving liquid path switching, allowing the calibration liquid and fluid samples at different testing stages to flow into the detection area and waste liquid area through different paths.

The detailed switching operation will be described in detail later.

The detection area, the waste liquid area and the liquid path control area are generally located between the first side and the second side of the test card 2.

Each functional area and the internal liquid path of the test card 2 have a plurality of implementation modes, and one of the preferred implementation modes is provided as follows.

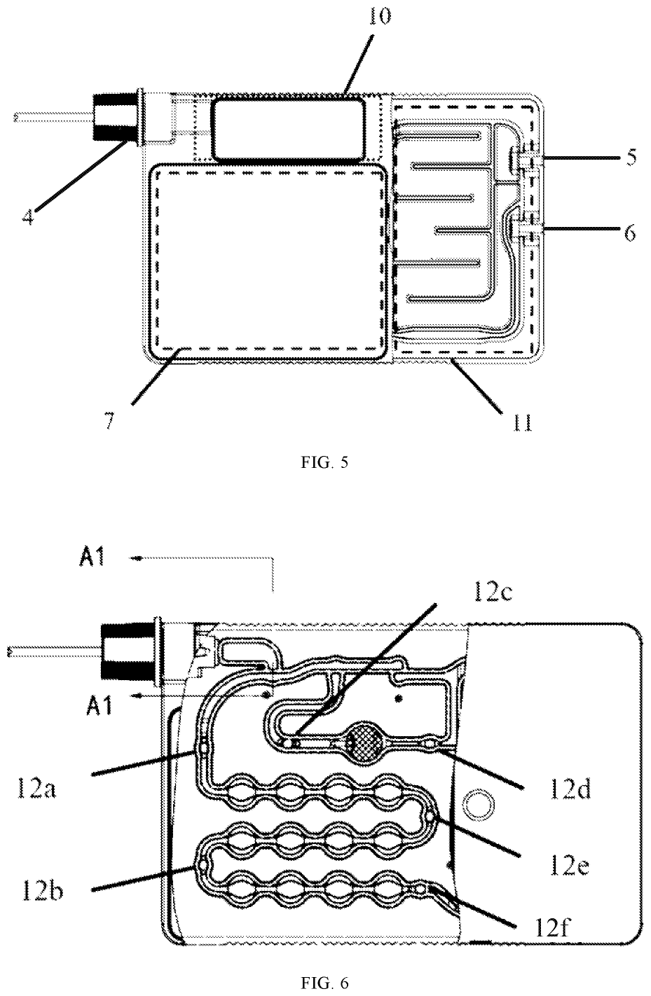

As shown in FIGS. 4-6, the removable test card 2 includes a card body, at least one portion of the card body is transparent. The card body may be made of molded plastic, additional material, or sets of materials. In order to better reflect the division of the transparent housing and internal functional areas of the card body, the semi-sectional views in FIGS. 4-6 are used to show the housing and internal functional areas of the test card 2.

The fluid sample to be detected is a blood sample. In an embodiment, the fluid sample is a whole blood sample without hemolysis. The detection area includes a blood gas detection area 7 and a hemoglobin and its derivatives detection area 8. The waste liquid area 11 is configured to store the detected blood sample. The internal liquid path 9 is divided into a main liquid path and three controllable liquid paths. The main liquid path is connected to the calibration liquid inlet 6, the blood gas detection area 7 and the liquid path control area 10. In an embodiment, the blood gas detection area 7 is between the calibration liquid inlet 6 and the liquid path control area 10. The first controllable liquid path is configured to control the on-off of the liquid path between the sample inlet 4 and the main liquid path. The second controllable liquid path is configured to control the on-off of the liquid path between the main liquid path and the inlet of the hemoglobin and its derivatives detection area 8, and the outlet of the hemoglobin and its derivatives detection area 8 is communicated to the waste liquid area 11. The third controllable liquid path is configured to on-off the waste liquid area 11 and the main liquid path. The waste liquid area 11 is connected to the exhaust port 5. The blood gas control area and the second controllable liquid path are arranged with several position detection points. The liquid path control area 10 is provided with three valves 10a to 10c for controlling the on-off of the internal liquid path 9, respectively configured to control the first, second and third controllable liquid paths. The control mode will be described in detail in the following sections.

The blood gas detection area 7 has twelve sensor cavities defined as 7A to 7L, the cavities are sequentially arranged on the main liquid path. The cavity may be of various shapes, and the shapes of the cavities can be the same or different. However, in the flow direction of the liquid path, the width of the sensor cavity is wider than the width of the liquid path. Various types of sensors can be placed in the cavity. The twelve sensor cavities are arranged from far to near according to the distance from the calibration liquid inlet in the direction of the flow path, sequentially numbered 7A-7L. The first eleven sensor cavities 7A-7K are sequentially provided with different photochemical sensors, the twelfth sensor cavity 7L is used as a standby for future detection parameter expansion, and the hemoglobin and its derivatives detection area 8 is only a cavity and is not provided with a sensor.

In an embodiment, the photochemical sensors in each of the first ten sensor cavities 7A-7J are configured to detect the blood gas parameters in the blood fluid sample, such as CO₂, O₂, pH, Na⁺, M⁺⁺, or the like.

In an embodiment, the fluid sample can be a whole blood sample, a urine sample and other types of human body fluid samples, and in this case, the sensor in the test card 2 detects corresponding biochemical parameter indexes.

The control mode of the test card 2 is described in detail below.

Before detecting the blood samples, it is necessary to calibrate all sensors in test card 2, that is, inject calibration liquid into the sensor, empty all calibration liquid in test card 2 after calibration, and then inject fluid samples to be detected. In an embodiment, the fluid samples are blood samples that do not require hemolysis. The testing is completed in the detection area, and the corresponding parameters are read through the host of the in-vitro medical diagnosis system.

After completing the diagnostic analysis, the corresponding parameters and diagnostic results are displayed on the host screen, so that medical personnel can timely learn about the corresponding situation.

The specific operation steps are as follows:
operation S1: placing the test card 2 into the first area 1a;
operation S2: pumping the calibration liquid into the detection area in the test card 2;
operation S3: after finishing calibration, transferring the calibration liquid in the test card 2 to the waste liquid area 11 in the test card 2;
operation S4: injecting blood sample into the detection area to complete the detection of at least hemoglobin and blood gas in the detection area; and
operation S5: transferring the blood sample in the detection area to the waste liquid area 11 in the test card 2 to complete the detection.

The operation S1 specifically includes as follows. Before the test card 2 is jointed with the reagent pack 3 and the host 1, the test card 2 is internally dry and has no calibration liquid. The calibration liquid is stored in the reagent pack 3, separated from the test card 2. The detection position is the first area 1a in the host 1. When the test card 2 is placed in the first area 1a, the fluid sample in the test card 2 is detected. As shown in FIG. 6, at least six position detection points 12a to 12f are arranged in the test card 2 and can receive light intensity emitted by a detection light source and transmitted through the test card 2, so that whether liquid exists at the position detection points 12a to 12f or not can be detected and judged.

The operation S2 specifically includes as follows. The third controllable liquid path is communicated, the first and second controllable liquid paths are controllably closed. The host 1 controls the peristaltic pump 16 and the three-way valve 17 in the reagent pack 3, switching the three-way valve 17 to the calibration liquid pipeline 19, pumping the calibrating liquid (i.e., standard sample liquid with known composition and concentration) in the calibration liquid bag 15 into the test card 2 through a connecting piece 20 inserted into the calibrating liquid inlet of the test card 2. When the position detection point 12a detects that liquid exists, the host 1 controls the peristaltic pump 16 in the reagent pack 3 to stop moving, at the moment, the sensor cavities 7A to 7K are filled with the calibration liquid. The host 1 starts calibrating the sensors (i.e., reading the readings of the liquid sensors of the standard sample measured by the sensors), and the host 1 finishes calibrating after reading the detection values of the sensors.

The operation S3 specifically includes as follows. The third controllable liquid path is kept communicated, and the first and second controllable liquid paths are kept closed. The host 1 controls the three-way valve 17 in the reagent pack 3 to be switched to the air channel. The peristaltic pump 16 is controlled to pump the air into the test card 2 through the connecting piece 20 inserted into the calibration liquid inlet of the test card 2. Because the waste liquid area 11 of the test card 2 is connected to the outside air through the exhaust port 5, with the pumping of the air, the calibration liquid continuously moves to the waste liquid area 11 in the first and third liquid paths until all the calibration liquid enters the waste liquid area 11.

The operation S4 includes two stages, i.e., a first stage and a second stage as follows.

The blood gas detection is performed in the first stage. Specifically, the first controllable liquid path is communicated, the second and third controllable liquid paths are closed, and the blood gas detection is performed in this case. The blood sample is injected from the sample inlet 4 of the test card 2, without hemolysis, or the host 1 controls the peristaltic pump 16 to rotate reversely, extracting the air in the test card 2, negative pressure is generated in the test card 2 to suck blood at the sample inlet 4 into the test card 2. Thus, after it is determined by the position detection points 12a to 12f that the blood sample to be detected completely enters the sensor cavities 7A to 7L, stopping injecting the blood sample to be detected into the test card 2, alternatively, the host 1 controls the peristaltic pump 16 to stop pumping air, and the blood sample is detected by using the sensor cavities 7A to 7L. In an embodiment, the photochemical sensors are arranged in the sensor cavities 7A to 7K, the sensor cavity 7L is used as standby sensors or other types of sensors are placed in the sensor cavity 7L.

The basic detection principle of the photochemical sensor is that the photochemical method uses organic dyes to emit fluorescence with different wavelengths from the irradiation light under specific wavelengths of light, influenced by substances such as O₂ and CO₂ concentration and pH. The fluorescence is transmitted to the detector to detect its fluorescence signal and perform quantitative analysis, thereby being able to detect O₂, CO₂, and pH values.

Entering the second stage after completing blood gas detection, that is, hemoglobin and its derivatives are detected. The first controllable liquid path is kept communicated and the third controllable liquid path is kept closed, the second controllable liquid path is opened. The host 1 controls the peristaltic pump 16 to rotate forward, pumping air into the test card 2, so that the blood in the blood gas detection area is sent into the hemoglobin and its derivatives detection area 8 through the second controllable liquid path. Then the host 1 controls the peristaltic pump 16 to stop pumping air into the test card 2, using the light emitted from the first light source to irradiate the hemoglobin and its derivatives detection area 8 from one side, and receiving the transmitted light from the other side of the hemoglobin and its derivatives detection area 8 to detect whether hemoglobin and its derivatives are present in the blood sample or not according to the detection principle of colorimetric method.

The operation S5 specifically includes as follows. After completing the detection of hemoglobin and its derivatives, the host 1 controls the peristaltic pump 16 to rotate forward and pumps air into the test card 2 to push the blood sample in the hemoglobin and its derivatives detection area 8 into the waste liquid area 11. After the blood sample enters the waste liquid area 11, the host 1 controls the peristaltic pump 16 to stop rotating and the test is finished.

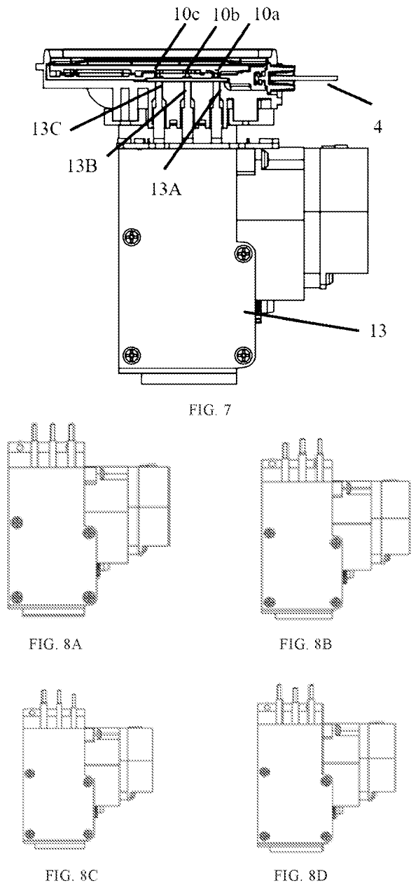

In the above operation steps, the control of the first, second and third controllable liquid paths is realized as shown in FIG. 7 and FIG. 8A-8D.

Three valves 10a to 10c are inside of the liquid path control area 10. The valves 10a to 10c are respectively configured to control the on-off of the first, second and third controllable liquid paths, and the on-off of the valves 10a to 10c are respectively driven by the control mechanisms 13A to 13C in the valve control device 13. When one of the control mechanisms 13A to 13C moves downwards, one corresponding valve in the valves 10a to 10c is opened, and the corresponding controllable liquid path is conductive. Otherwise, when one of the control mechanisms 13A to 13C moves upwards to the end, one corresponding valve in the valves 10a to 10c is closed, and the corresponding controllable liquid path is not conductive.

Specifically, in the operation S1, the valves 10a to 10c are all in an off state, and the positions of the control mechanisms are shown in FIG. 8A, that is, the first, second and third controllable liquid paths are all disconnected.

In the operations S2 and S3, the valves 10a to 10b are in a closed state, the valve 10c is in a conductive state, the positions of the control mechanisms are shown in FIG. 8B, and the main liquid path is connected to the waste liquid area 11, so that the calibration liquid can be pumped into the test card 2. After completing calibration, air is pumped into the test card 2, so that the calibration liquid enters the waste liquid area 11.

In the first stage of the operation S4, the valve 10a is controlled to be conductive, the valves 10b to 10c are in the closed state, the positions of all the control mechanisms are shown in FIG. 8C, and the main liquid path is connected to the sample inlet 4, so that the blood sample can be injected into the test card 2, and the sample blood reaches the blood gas detection area 7 for blood gas detection.

In the second stage of operation S4, that is, after the blood gas detection is completed, the valve 10b is controlled to be conductive, and the valves 10a and 10c are in the off state. The positions of the control mechanisms are shown in FIG. 8D, the main liquid path is connected to the hemoglobin and its derivatives detection area 8, so that the blood sample can enter the hemoglobin and its derivatives detection area 8, and the detection of the hemoglobin and its derivatives is performed.

After the detection is completed, the blood sample is sent into waste liquid area 11.

The turn-off sequence of the valves 10a to 10c may be set to a fixed timing sequence or customized and logically programmed according to the needs of the operator.

In an exemplary embodiment, the widths of the first, second and third liquid paths may be set to be different. In an embodiment, the width of the second liquid path is greater than the width of the first liquid path. The thickness of the sensor cavity of the test card 2 in the blood gas detection area 7 is different from the thickness of the cavity in the hemoglobin and its derivatives detection area 8, so as to meet the requirements of blood gas photochemical detection of whole blood sample and different detection methods of hemoglobin and its derivatives.

In an exemplary embodiment, the test card 2 is configured to be discarded after using. Alternatively, the test card 2 can be configured to be recycled to test more than one fluid sample.

Furthermore, an electrochemical sensor can be used in the test card 2 to detect blood gas, hemoglobin and its derivatives. The electrochemical detection technology is mature, which is not required to be described in detail.

Reagent Pack

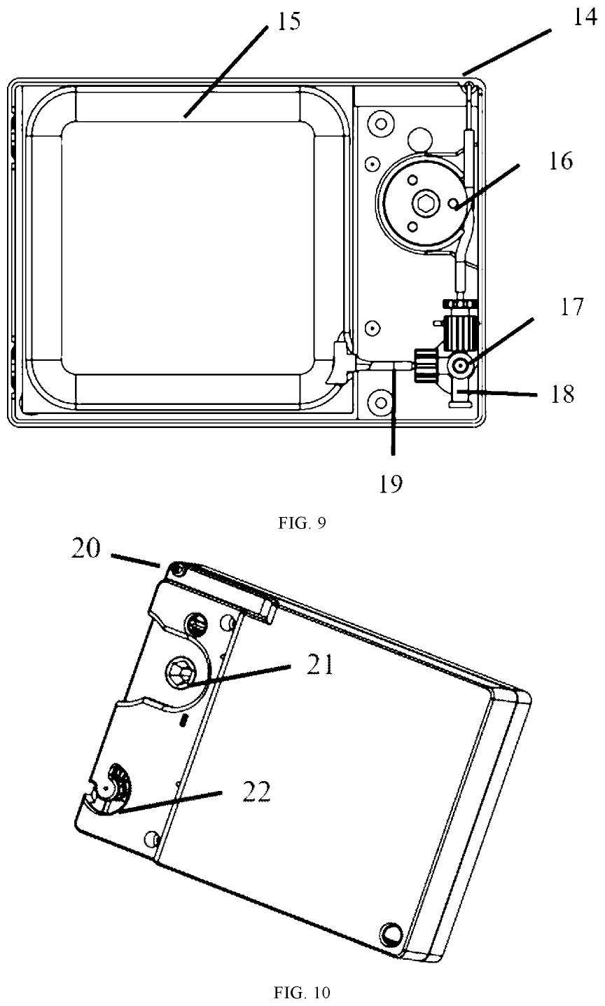

As shown in FIG. 9, FIG. 9 is a schematic cross-sectional view of the reagent pack 3. The reagent pack 3 may be disposable and removable. The reagent pack 3 includes a housing 14, a calibration liquid bag 15, a peristaltic pump 16, a three-way valve 17, an air pipeline 18, and a calibration liquid pipeline 19, a connecting piece 20, a pump interface 21, and a valve connecting piece 22. The calibration liquid bag 15, the peristaltic pump 16, the three-way valve 17, the air pipeline 18, and the calibration liquid pipeline 19 are located in the housing 14. The connecting piece 20 is inserted into the calibration liquid inlet 6 of the test card 2. The pump interface 21 is connected to the shaft of the stepper motor of the host 1. The stepper motor drives the peristaltic pump 16 of the reagent pack 3 to forward rotate or reversely rotate through the pump interface 21. The host 1 controls the three-way valve 17 of the reagent pack 3 to switch through the valve connecting piece 22, thereby connecting the pump interface 21 to the air pipeline 18 or the calibration liquid pipeline 19. The calibration liquid bag 15 is configured to store the calibration liquid and is configured to be connected to the three-way valve 17 through the calibration liquid pipeline 19.

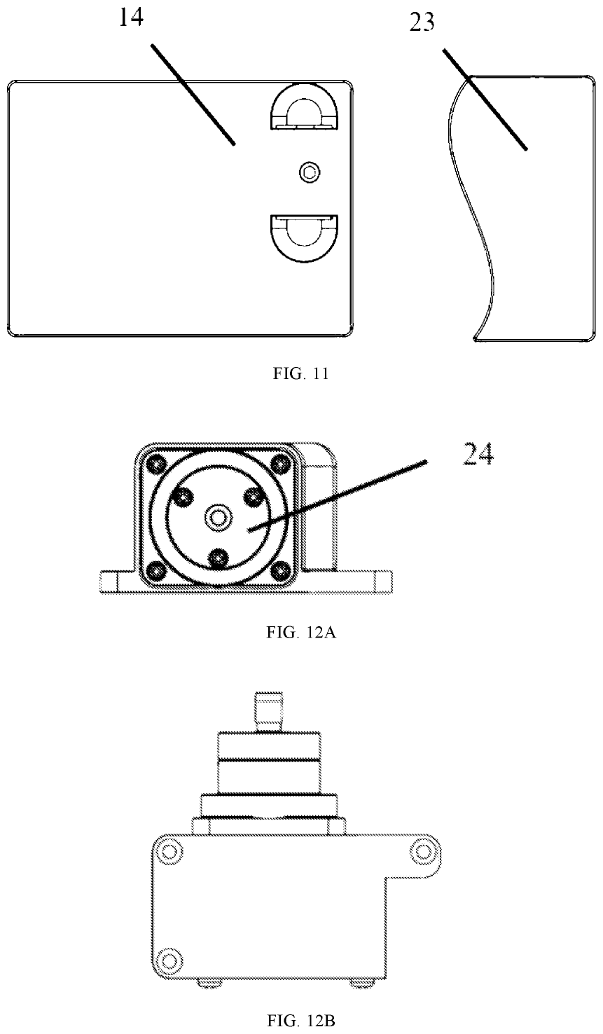

In an exemplary embodiment, the housing 14 of reagent pack 3 is made of plastic, and can also be made of other materials or sets of materials. The reagent pack 3 can also include a decoration cover. As shown in FIG. 11, the decoration cover 23 of the reagent pack 3 is connected to the front part of the housing 14 of the reagent pack 3. When reagent pack 3 is not in use (i.e., not connected to the host 1), the decoration cover 23 of the reagent pack 3 can protect the housing 14 of reagent pack 3. The calibration liquid bag 15 can be an unused soft elastic fluid bag filled with the calibration liquid.

Before the test card 2 and the reagent pack 3 are respectively jointed with the host 1, the interior of the test card 2 is dry and has no calibration liquid, and the calibration liquid is completely stored in the reagent pack 3 and is separated from the test card 2.

When the test card 2 is placed in the first area 1a, the connecting piece 20 on the reagent pack 3 is inserted into the calibration liquid inlet 6 of the test card 2. In an embodiment, the connecting piece 20 is a tubular steel needle, and the circumference of the tubular steel needle is provided with a sealing ring, such as a rubber sealing ring, in order to ensure the sealing performance of the inserting position of the tubular steel needle and the calibrating liquid inlet 6 of the test card 2. After the calibration liquid is pumped into the test card 2, the calibration liquid cannot leak from the calibration liquid inlet 6. The controller in the host 1 controls the rotating shaft of the stepper motor to output power, and the peristaltic pump 16 in the reagent pack 3 is controlled to forward rotate or reversely rotate through the pump interface 21, so that the test card 2 can complete calibration and fluid sample detection, and the specific working principle is described in detail below.

When the test card 2 and the reagent pack 3 are respectively arranged in the first area 1a and the second area 1b of the host 1, the working principle of the reagent pack 3 is as follows.

When the calibration liquid in the reagent pack 3 needs to be output, such as, in the calibration stage of the test card 2 in the operation S2, the three-way valve 17 in the reagent pack 3 is switched to the calibration liquid pipeline 19 to be connected to the connecting piece 20. The peristaltic pump 16 is in a forward rotation state, so that the reagent pack 3 can output the calibration liquid outwards, and after the calibration liquid enters the test card 2, the photochemical sensor in the test card 2 is calibrated.

When air needs to be pumped to the outside, for example, when the calibration liquid and the detected blood sample are transferred to the waste liquid area 11 of the test card 2 in the operation S3 and the operation S5, or when the blood sample is transferred to the hemoglobin and its derivatives detection area 8 after the blood gas detection is completed in the second stage of operation S4, the three-way valve 17 in the reagent pack 3 is switched to the air pipeline 18 to be connected to the connecting piece 20, the peristaltic pump 16 is in the forward rotation state, so that the reagent pack 3 can pump air into the test card 2, and the blood sample in the test card 2 can flow in the liquid path conducted in the test card 2 with the pumping of the air.

When air needs to be sucked into the reagent pack 3, for example, in the first stage of the operation S4, when the blood sample is sucked into the blood gas detection area 7 in the test card 2, the three-way valve 17 in the reagent pack 3 is switched to the air pipeline 18 to be connected to the connecting piece 20. The peristaltic pump 16 is in a reverse rotation state, so that the air in the test card 2 is sucked into the reagent pack 3, and the blood sample enters the liquid path in the test card 2 under the action of air pressure, thereby performing the blood gas detection.

Host

As previously described, the host 1 includes the housing, and the processing circuitry, the power supply circuitry, and the optical element are located in the housing. The housing may be plastic or any other material suitable for use in the present disclosure. The housing includes the first area 1a configured to at least partially accommodate the removable test card 2, and the second area 1b configured to at least partially accommodate the removable reagent pack 3. The removable test card 2 is jointed with the host 1 and the removable reagent pack 3 through the first area 1a and the second area 1b, respectively, thereby performing blood gas detection, hemoglobin and its derivatives detection or other biochemical parameter detection. The test card 2 and the host 1 only have mechanical transmission connection, no liquid path connection. Only mechanical power transmission connection exists between the reagent pack 3 and the host 1, there is no liquid path connection between the reagent pack 3 and the host 1. The test card 2 is connected to the reagent pack 3 through the calibration liquid inlet 6, and calibration liquid flowing and air flowing are achieved. Due to above design mode, a liquid path system does not exist in the host 1, and liquid path flowing with the test card 2 and the reagent pack 3 is not needed.

In an exemplary embodiment, the first area 1a of the housing includes a test slot for accommodating the test card 2 containing the fluid sample, such as the blood sample. The syringe is configured to be connected to the sample inlet 4 of the test card 2. The host 1 is configured to test the fluid sample and report the result to the user through the output unit, for example, host 1 may include a display serving as the output unit. However, in this embodiment or other embodiments, diagnostic results may also or alternatively be reported to the user by other output units, including an audio output unit, a data communication output unit, or a print output unit, and so on.

In an exemplary embodiment, once the fluid sample is tested, the test card 2 may be removed from the host 1. The host 1 may include an ejection button, once the test is complete, the user can depress the ejection button to eject the test card 2 from the test slot. When the test cycle is completed, the host 1 may also be configured to automatically eject the test card 2. In an embodiment, the host 1 may be portable.

In an exemplary embodiment, the diagnostic results are displayed on the display. The processing circuitry of host 1 may cause the display to show information relating to a particular application. The display may be a unidirectional screen configured to display the output to the user, alternatively, the display may be a touch screen configured to receive and respond to the user's touch input. In an exemplary embodiment, the diagnostic device further includes a print slot configured to receive paper output by the printer housed within the host 1.

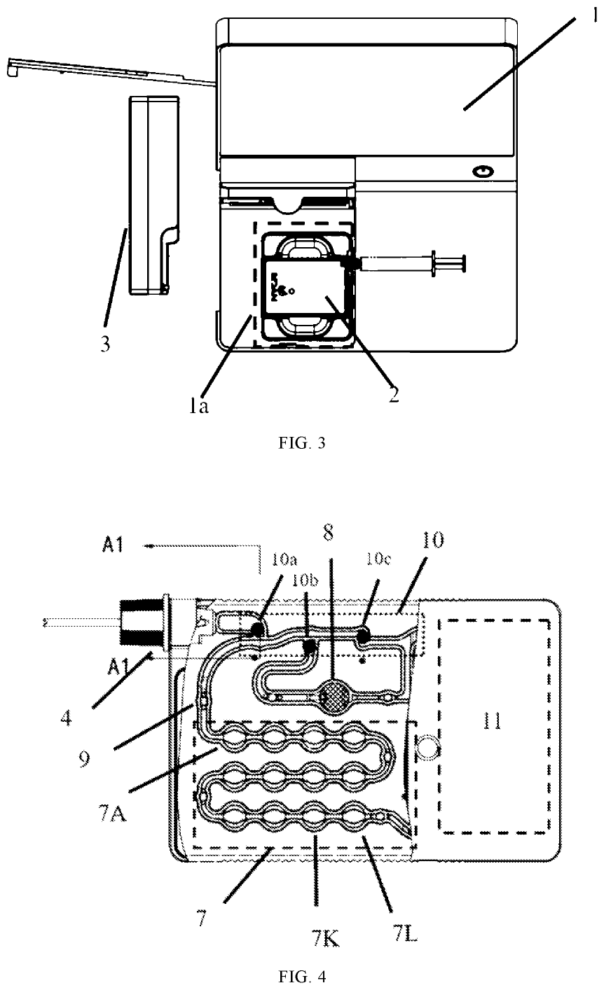

In an exemplary embodiment, as shown in FIG. 3, the second area 1b of the host 1 includes a reagent pack door configured to be opened from a side of the host 1. An opening behind the reagent pack door and the reagent pack door are configured to accommodate the reagent pack 3. The reagent pack door can be opened by a latch, or other mechanisms in another exemplary embodiment. The reagent pack door can also be located at other positions, for example, the reagent pack door may be located on either side, back, front or top of the host 1. The latch is adjacent to the reagent pack door for positioning. The power supply interface for supplying power to the pump and the power supply interface for supplying power to the valve components are set outside of the reagent pack.

In an exemplary embodiment, except for the connecting piece 20 of the reagent pack 3 directly connected to the calibration fluid inlet 6 of the test card 2, the reagent pack 3 and the host 1 can also be combined through the following methods. The connecting piece 20 of reagent pack 3 is connected to the host sample inlet above one side of host 1, and the host sample inlet is further connected to the calibration liquid inlet 6 of test card 2 to ensure that the calibration liquid can flow into the test card 2. The calibration liquid bag 15 of reagent pack 3 includes a bayonet groove, and the bayonet groove is configured to clamp onto the fixing device of reagent pack 3 on one side of host 1, in order to fix the reagent pack 3 in the second area 1b of host 1. The valve connecting piece 22 of reagent pack 3 is connected to the on-off valve control device of the reagent pack 3 on one side of host 1, thereby achieving on-off control of the three-way valve 17 of the reagent pack 3 by host 1. In addition, the pump interface 21 of reagent pack 3 is connected to the peristaltic pump control device on the side of host 1, such as the output shaft of the stepper motor, so that the host 1 can control the peristaltic pump 16 in the reagent pack 3.

In an exemplary embodiment, the host 1 may include one or more ports configured to accommodate a cable or other connection mechanism. The port can be configured to connect the host 1 to other pieces of the system (such as a communication network) or to upload or download information to the host 1. The host 1 can also be configured to exchange data wirelessly, including through Wi Fi (wireless internet technology), another wireless internet connection, or any other wireless information exchange. The host 1 can also include speakers configured to transmit noise or sound response to the user. The host 1 can also include a handle configured to hold the host 1. The handle rotates between two positions depending on whether it is used or not. In exemplary embodiments, the host 1 may also include a support leg configured to allow the host 1 to place on a desktop or other surface. In an exemplary embodiment, the host 1 may also include a barcode scanner installed on the side of host 1. The barcode scanner is configured to scan barcodes on the test card 2, calibration liquid bag 15, or any other items that have a scannable barcode and are used on the host 1. Barcode scanners can also be configured to scan barcode labels that represent patient or operator identities. In an exemplary embodiment, the barcode scanner emits a beam of light covering the barcode. When the barcode is successfully scanned, the host 1 emits a beep sound and the beam is automatically turn off. When the barcode is not successfully scanned, the host 1 alert the user by making noise or on the display through some other output unit. In exemplary embodiments, the barcode scanner is a one-dimensional barcode scanner. In other embodiments, the barcode scanner is a two-dimensional scanner.

According to an exemplary embodiment, the processing circuit of the host 1 includes an analog-to-digital converter and an analog control board. The analog-to-digital converter is configured to process an analog signal from the photochemical sensor, and transmits the processed digital signal to the analog control board. When the analog control boards herein and in the drawings are designated as “analog control board”, the analog control board may include digital processing. Furthermore, the analog control board may utilize the digital-to-analog converter to convert the digital output (turn on/turn off modulated signal) to the analog signal (e.g., for the photochemical sensor).

The processing circuitry and power supply circuitry in the host 1 may be used as independent printed circuit board (PCB), integrated on the same PCB, or combined with integration and distribution. The processing circuit and the power supply circuit may include discrete components and/or integrated circuits. For example, the power supply circuit can include all discrete electronic components. The processing circuit may include one or more processors. The processor can be operated in multiple ways as a general-purpose processor, a specialized integrated circuit (ASIC), one or more field programmable gate arrays (FPGA), a set of processing components, or other suitable electronic processing components. The processing circuit may also include one or more memories. The memory can be one or more devices used to store data and/or computer code. The memory can be or include non-transient volatile memory and/or non-volatile memory. The memory may include a database component, an object code component, a script component, or any other type of information structure used to support various activities and the information structure described herein. The memory can be propagatively connected to the processor and includes computer code modules for executing one or more programs described herein.

The optical elements of the host 1 now are described in detail as previously described. In embodiments of the present disclosure, the blood gas parameters in the blood sample are measured by the photochemical sensor, and hemoglobin and its derivatives are detected using optical methods, such as colorimetry without the photochemical sensor. Thus, optical elements are needed to provide the light source, excite the photochemical sensor, transmit the optical signals, and the like.

Specifically, the optical element of the host 1 includes two parts. The first part is arranged in the host 1, and the first part includes a first light source for detecting the hemoglobin and its derivatives and a second light source for detecting the fluid position of the test card 2. The different detection beams are emitted to detect the presence or absence of fluid in the hemoglobin and its derivatives of the blood sample and in each position monitoring point of the test card 2, respectively. The second part is arranged in the host 1, and equipped with a light source 24 for optically detecting the blood gas component of the blood sample, as shown in FIG. 12A and FIG. 12B. Specifically, the light source 24 is an excitation light source 24 configured to emit an excitation beam to excite the photochemical sensor in the test card 2, and the excitation light source is located within the host 1 and is lower in height than the first area 1a.

In an exemplary embodiment, the diagnostic device includes the valve control mechanism configured to control the valve component of the test card 2.

The beneficial effect of the invention lies in, there is no liquid path in the host of the in-vitro diagnostic system, therefore, there is no hidden danger of liquid path leakage. At the same time, there is no calibration liquid in the test card, reduces the requirements for the storage condition and environment of the test card in the transportation and storage links of the test card. Meanwhile, the detection of blood gas, hemoglobin and derivatives thereof can be realized in a single test card, so that medical resources are greatly saved, the diagnosis cost is reduced, repeated blood collection is not needed, the acquisition speed of diagnosis results is accelerated, and the medical experience of a patient is remarkably improved. Simultaneously, an optical system inside of the host is compact in structure and small in optical path conduction loss, and particularly, blood gas, hemoglobin and derivatives thereof can be accurately detected through an optical method, so that the accuracy of in-vitro diagnosis is further improved.

The terms “generally”, “about”, “substantially”, and similar terms used here have a broad meaning consistent with the generally accepted usage recognized by those skilled in the art, and the subject of this disclosure belongs to the generally accepted usage. Those skilled in the art who review this disclosure should understand that these terms are intended to allow descriptions of certain features described and claimed, without limiting the scope of these features to a set precise numerical range. Therefore, these terms should be interpreted as non-substantive or incoherent modifications or modifications of the subject matter described and claimed priority should be considered within the scope of the present disclosure and explained by the accompanying claims. It should be noted that the term “exemplary” used in this disclosure to describe different embodiments refers to possible examples, illustrations representing and/or possible embodiments (this term does not imply that such embodiments must be extraordinary examples or highest-level examples). The terms “coupled” and “connected” and their analogues used in this disclosure refer to the direct or indirect combination of two components with each other. This combination can be static (such as permanent) or movable (such as removable or releasable). This combination can be achieved by combining two components or integrating the terrain into a single entity with any additional intermediate components, or by combining two components or two components attached to each other with any additional intermediate components. The structure and arrangement of the system and the methods for providing in-vitro medical diagnostic devices as shown in various exemplary embodiments are just illustrated.

Only some embodiments have been described in detail in present disclosure, but it will be readily appreciated by those skilled in the art with reference to this disclosure, there may be many modifications (e.g., variations in size, structure, shape and scale of various elements, parameter values, mounting arrangements, use of materials, color, changes in orientation, and so on.) without substantially departing from the novel teachings and advantages of the subject matter disclosed herein. For example, the components shown as a whole can be composed of multiple parts or components, and the position of the components can be reversed or otherwise changed, as well as the nature, quantity, or position of discrete components can be changed. All such modifications are intended to be included in the scope of the present disclosure as defined in the appended claims. The order or sequence of any process or method step can be changed or reordered according to alternative embodiments. The design, operating conditions, and arrangement of various exemplary embodiments can be replaced, modified, altered, and omitted without departing from the scope of the present disclosure.

The diagnostic device is generally shown to include the processing circuitry including the memory. The processing circuitry may include the processor. The processor can be operated as a general-purpose processor, a specialized integrated circuit (ASIC), one or more field programmable gate arrays (FPGA), a set of processing components, or other suitable electronic processing components. The memory can be one or more devices configured to store data and/or computer code (e.g., RAM, ROM, flash memory, hard disk memory, or the like), to complete and/or implement various programs described in present disclosure. The memory can be or include non-transient volatile memory and/or non-volatile memory. The memory may include the database component, the object code component, the script component, or any other type of information structure used to support various activities and the information structure described herein. The memory can be propagatively connected to the processor and includes computer code modules for executing one or more programs described herein. The foregoing is merely a preferred embodiment of the present disclosure, without limiting the disclosure, any modifications, equivalents, substitutions, and modifications made within the spirit and principles of the disclosure should be included within the scope of the disclosure.

What is claimed is:

1. An in-vitro medical diagnostic device, comprising:
a housing;
a first mounting area configured to mount a removable reagent pack;
a second mounting area configured to mount a removable test card;
an optical system configured for in-vitro diagnosis and being inside of the device; and
a power drive system configured to control at least on-off of a pipeline in the removable test card and/or to control a flow of a liquid in the removable reagent pack, wherein the power drive system is inside of the device, and the optical system is at least configured to detect a blood gas, a hemoglobin and/or an electrolyte in the removable test card.

2. The in-vitro medical diagnostic device as claimed in claim 1, further comprising a positioning attachment area configured to connect an interface of the removable reagent pack and an interface of the removable test card.

3. The in-vitro medical diagnostic device as claimed in claim 1, wherein the optical system has one or more light source systems, wherein at least one first light source system configured to activate a photochemical sensor in the removable test card is disposed below the second mounting area; an excitation light emitted by the first light source system passes through a first optical path and enters one end of an optical fiber bundle, and the other end of the optical fiber bundle is provided with an optical element configured to focus and illuminate the photochemical sensor in the removable test card; the first optical path comprises at least a first lens, a first filter, and a first beam splitter; the fiber optic bundle receives a fluorescence and/or reflected light generated in the removable test card, and transmits the fluorescence and/or reflected light to an optical detection element through a second optical path; and the second optical path is at least partially different from the first optical path.

4. The in-vitro medical diagnostic device as claimed in claim 3, further comprising at least one second light source system disposed above the second mounting region and configured to detect the hemoglobin and derivatives thereof and/or detect a flow position of an internal liquid of the removable test card.

5. The in-vitro medical diagnostic device as claimed in claim 3, wherein an excitation light emitted from the first light source has a central wavelength of 450 nm to 470 nm.

6. The in-vitro medical diagnostic device as claimed in claim 4, wherein a central wavelength of the fluorescent and/or reflected light is 520 nm to 650 nm.

7. The in-vitro medical diagnostic device as claimed in claim 1, wherein the optical system comprises at least two light sources distributed on different sides of the second mounting area.

8. The in-vitro medical diagnostic device as claimed in claim 1, wherein the power drive system further comprises a first power part configured to control the on/off of the pipeline in the removable test card.

9. The in-vitro medical diagnostic device as claimed in claim 1, wherein the power drive system further comprises a second power part configured to control switching of the pipeline and liquid flow in the removable reagent pack.

10. The in-vitro medical diagnostic device as claimed in claim 9, wherein the second power part comprises a first motor configured to control switching of a three-way valve in the removable reagent pack and a second motor configured to control rotation of a peristaltic pump in the removable reagent pack.

11. The in-vitro medical diagnostic device as claimed in claim 1, wherein the power drive system further comprises a third power part configured to lock and unlock the removable test card.

12. An in-vitro medical diagnosis system, comprising:
a blood gas analyzer host;
a removable test card; and
a removable reagent pack, wherein the removable reagent pack and the removable test card are connected through respective interfaces, and calibration, sample detection and emptying are completed by providing power for the removable reagent pack and controlling on-off of a pipeline of the removable test card through the blood gas analyzer host.

13. The in-vitro medical diagnosis system as claimed in claim 12, wherein the removable test card comprises at least a blood gas detection area and a hemoglobin and derivatives detection area; and a detection in the hemoglobin and derivatives detection area is implemented by colorimetric, electrochemical, or hemolytic method.

14. The in-vitro medical diagnosis system as claimed in claim 12, wherein the removable reagent pack comprises at least one liquid storage device comprising an output pipeline, at least one transport control device configured to control at least a flow direction in a pipeline inside of the removable reagent pack, and at least one positioning mechanism configured to fix the removable reagent pack on the blood gas analyzer host, so that the transport control device is driven by the blood gas analyzer host.

15. The in-vitro medical diagnosis system as claimed in claim 13, wherein a pipeline between the blood gas detection region and the hemoglobin and derivatives detection area is on-off connectable.

16. The in-vitro medical diagnosis system as claimed in claim 12, wherein the device controls on-off of the pipeline of the removable test card through a power system such that at least blood gas detection and hemoglobin detection is performed on the same blood sample or on different blood samples.

17. The in-vitro medical diagnosis system as claimed in claim 12, wherein the removable reagent pack at least comprises calibration liquid required for one or more calibrations of the removable test card.

18. The in-vitro medical diagnosis system as claimed in claim 12, wherein an elastomeric material and/or a valve is inside or on a surface of the removable test card, and the elastomeric material and/or the valve is configured to implement an on-off connection of the pipeline at a corresponding position of the removable test card.

19. The in-vitro medical diagnosis system as claimed in claim 12, wherein the removable test card comprises at least three external interfaces; and the at least three external interfaces comprise a first interface configured to inject a calibration liquid, a second interface configured to inject a sample, and a third interface connected to a waste liquid area of the removable test card.

20. The in-vitro medical diagnosis system as claimed in claim 12, wherein the removable test card comprises at least three pipeline control parts; the at least three pipeline control parts are respectively configured to control an on-off connection of the pipeline between the blood gas detection area and the hemoglobin and derivatives detection area, the on-off connection between a sample inlet of the removable test card and a liquid path inside of the removable test card, and the on-off connection between the liquid path inside of the removable test card and the waste liquid area of the removable test card; and an electrical coupling connection is between the blood gas analyzer host and the removable test card, and the electrical coupling connection is configured to supply power to an electrochemical sensor in the removable test card by the blood gas analyzer host.
